(19) United States
(12) Patent Application Publication
(10) Pub. No.: US 2018/0235445 A1
(43) Pub. Date: Aug. 23, 2018

(54) ENDOSCOPE PROCESSOR AND ENDOSCOPE CONNECTION SYSTEM

(71) Applicant: HOYA CORPORATION, Tokyo (JP)

(72) Inventors: Hiroki SASAMURA, Tokyo (JP); Shotaro KOBAYASHI, Tokyo (JP); Masahiro KOMATSU, Tokyo (JP); Kunihiko ONOBORI, Tokyo (JP)

(73) Assignee: HOYA CORPORATION, Tokyo (JP)

(21) Appl. No.: 15/753,060

(22) PCT Filed: Sep. 30, 2016
(86) PCT No.: PCT/JP2016/079877

§ 371 Date: Feb. 15, 2018

(30) Foreign Application Priority Data
Sep. 30, 2015 (JP) 2015-193208

(51) Int. Cl. A61B 1/00; G02B 23/24

(52) U.S. Cl. CPC A61B 1/00124; A61B 1/00029; A61B 1/00009; A61B 1/00006; G02B 23/2484

(57) ABSTRACT

An endoscope processor includes a connector configured to connect with a second connector, provided on an endoscope; a protective ground-contact connected to ground; and an electrostatic inductor connected to, and electrically conductive with, the protective ground-contact, the electrostatic inductor positioned in a connection space between the connector and the second connector when the endoscope and the endoscope processor are not connected to each other.

### TECHNICAL FIELD

The present invention relates to an endoscope processor and an endoscope connection system, and in particular, relates to an electrostatic protection mechanism.

### BACKGROUND ART

Patent Literature 1 discloses an electrostatic protection mechanism in an endoscope processor and an endoscope connection system for connecting an endoscope to a processor, in which an endoscope processor body is formed out of a primary ground member which is connected to ground, and a secondary ground member is provided on a connector of the endoscope and the endoscope processor, projects from the connector toward the primary ground member. Due to this electrostatic protection mechanism, when static electricity is generated on the endoscope with the endoscope processor connected to the endoscope, the electrical charge accumulated on the endoscope discharges from the secondary ground member to the primary ground member.

### CITATION LIST

### Patent Literature

Patent Literature 1: Japanese Unexamined Patent Publication No. 2014-18349

### SUMMARY OF INVENTION

### Technical Problem

However, in the endoscope connection system of Patent Literature 1, if a electrostatically-charged user's hand, etc., touches (or approaches closely to) the connector of the endoscope processor when the endoscope is not connected to the endoscope processor, an electrostatic charge discharges onto the connector, thereby risking an unintentional load on the electronic components (such as an IC) of the endoscope processor. In particular, due to the standards stipulated for medical devices which states that the patient circuit (relay processing circuit) of an endoscope processor must not be directly connected to ground, there is a tendency for electrostatic protection to be insufficient; hence, an effective countermeasure is required.

The present invention has been devised with consideration of the above-mentioned problems of the related art, and provides an endoscope processor and an endoscope connection system which can prevent electrostatic breakdown of the connector and/or the electronic component(s) (such as an IC) in the endoscope processor, even when the endoscope is not connected to the processor.

### Solution to Problem

The inventors of the present invention have devised the present invention upon focusing their attention on effective electrostatic protection (electrostatic blocking) of a connector of an endoscope processor when the endoscope is not connected to a processor.

According to an aspect of the present invention, an endoscope processor is provided, including a connector configured to connect with a second connector, the second connector provided on an endoscope; a protective ground-contact connected to ground; and an electrostatic inductor connected to, and electrically conductive with, the protective ground-contact, the electrostatic inductor positioned in a connection space between the connector and the second connector when the endoscope and the endoscope processor are not connected to each other.

When the connector of the endoscope processor and the second connector of the endoscope and are brought close to each other to be connected to each other, it is desirable for the electrostatic inductor to be configured to contact the second connector of the endoscope in the connection space before the connector and the second connector are connected to each other, and for the electrostatic inductor to be configured to continue to be in contact with the second connector of the endoscope upon the connector and the second connector being connected to each other.

It is desirable for the electrostatic inductor to be configured to continue to be in contact with the second connector of the endoscope by deforming upon the electrostatic inductor contacting with the second connector.

It is desirable for the electrostatic inductor to include a conductive elastic member extending from an outer periphery toward a center of the connector of the endoscope processor; and a parallel circuit including a capacitor and a resistor which are each connected between the conductive elastic member and the protective ground-contact.

It is desirable for the endoscope processor to include a patient circuit provided with at least one electronic component; and an impedance component connected between the patient circuit and the protective ground-contact. The endoscope processor is configured so that an accumulated electrostatic charge in the patient circuit discharges to the protective ground-contact via the impedance component. The impedance component is arranged so that the electronic component of the patient circuit avoids a discharge path that flows from the patient circuit to the protective ground-contact.

It is desirable for the endoscope processor to include a discharge resistor connected between the patient circuit and the protective ground-contact, the discharge resistor arranged in parallel with the impedance component.

It is desirable for the protective ground-contact to be provided on a body of the endoscope processor. The electrostatic inductor is provided on the protective ground-contact at a position immediately in front of the connector of the endoscope processor.

In an embodiment, an endoscope connection system is provided, configured to connect an endoscope to an endoscope processor via a connector and a second connector, wherein the endoscope processor includes a protective ground-contact connected to ground; and an electrostatic inductor connected to, and electrically conductive with, the protective ground-contact, the electrostatic inductor positioned in a connection space between the connector and the second connector when the endoscope and the endoscope processor are not connected to each other.

### Advantageous Effects of Invention

According to the present invention, an endoscope processor and an endoscope connection system is achieved, in which electrostatic breakdown of a connector and/or an electronic component(s) (such as an IC) in the endoscope processor can be prevented, even when the endoscope is not connected to the processor.

### BRIEF DESCRIPTION OF DRAWINGS

FIG. 1 shows a configuration of an endoscope.

FIG. 2 is a schematic diagram showing a configuration of an endoscope processor, to which a first embodiment of the present invention is applied.

FIG. 3 is a schematic diagram showing a configuration of an endoscope system (endoscope connection system) according to a first embodiment of the present invention.

FIG. 4 shows a front elevations view of an example of a configuration of a conductive elastic member (electrostatic inductor), as viewed in a direction facing the endoscope processor, in the opposite direction to the endoscope.

FIG. 5 shows a first diagram indicating a connecting operation of the endoscope system (endoscope connection system).

FIG. 6 shows a second diagram indicating a connecting operation of the endoscope system (endoscope connection system).

FIG. 7 shows a second embodiment of an endoscope system (endoscope connection system) according to the present invention.

FIG. 8 shows a third embodiment of an endoscope system (endoscope connection system) according to the present invention.

FIG. 9 shows a fourth embodiment of an endoscope system (endoscope connection system) according to the present invention.

FIG. 10A shows a fifth embodiment of an endoscope system (endoscope connection system), according to the present invention, in a state where an endoscope is not connected to an endoscope processor.

FIG. 10B shows the fifth of the endoscope system (endoscope connection system) of FIG. 10A, in a state immediately before the endoscope is connected to the endoscope processor.

### DESCRIPTION OF EMBODIMENTS

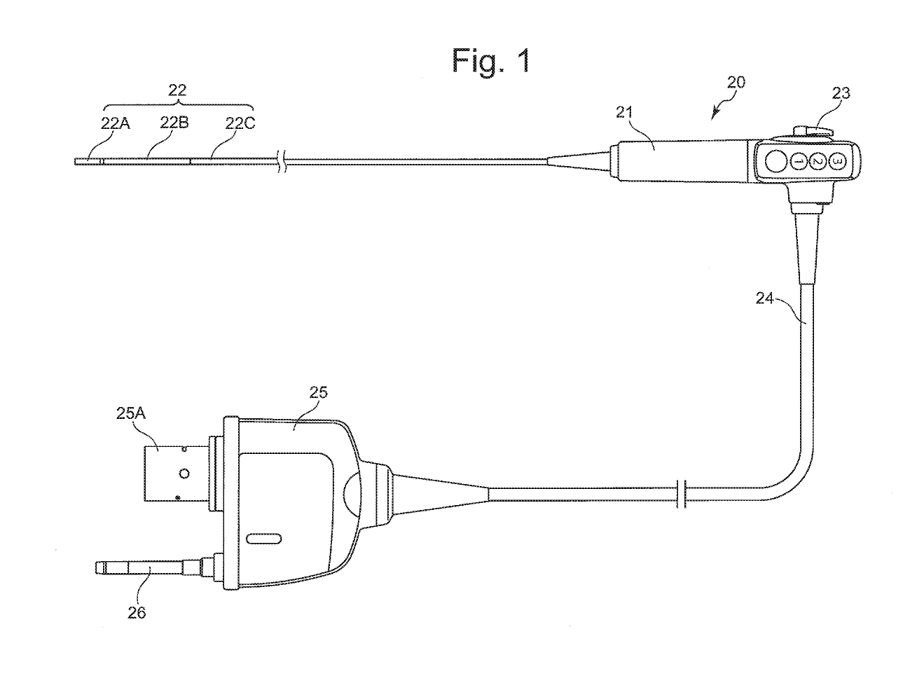

An endoscope system (endoscope connection system) 10 of each illustrated embodiment will be herein described with reference to FIGS. 1 through 10B. The endoscope system 10 is provided with an endoscope 20 configured to obtain an observation image (examination image) of an examinee (patient), and an endoscope processor 30 in which image processing is carried out on the observation image obtained by the endoscope 20.

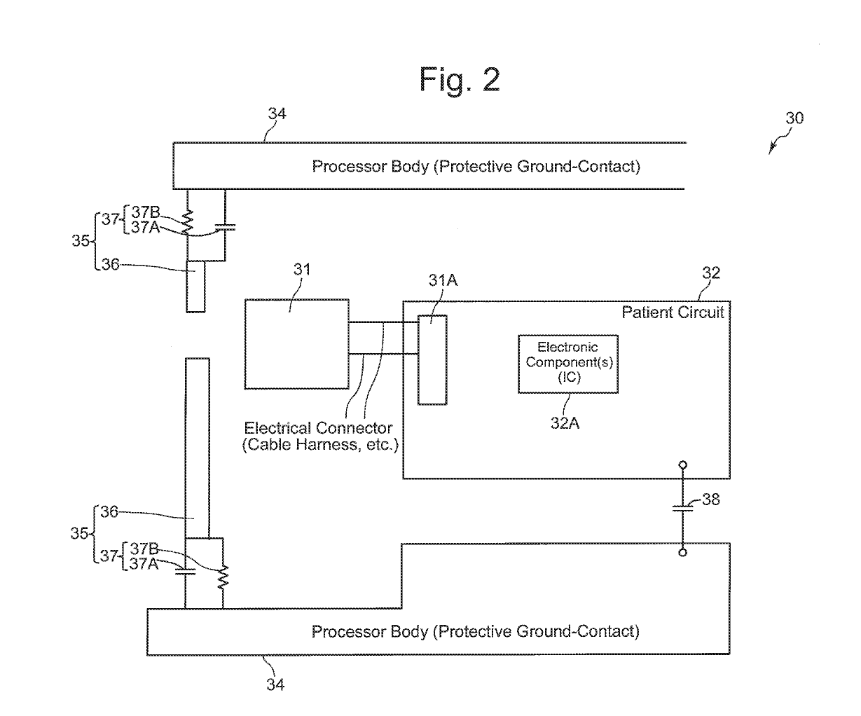

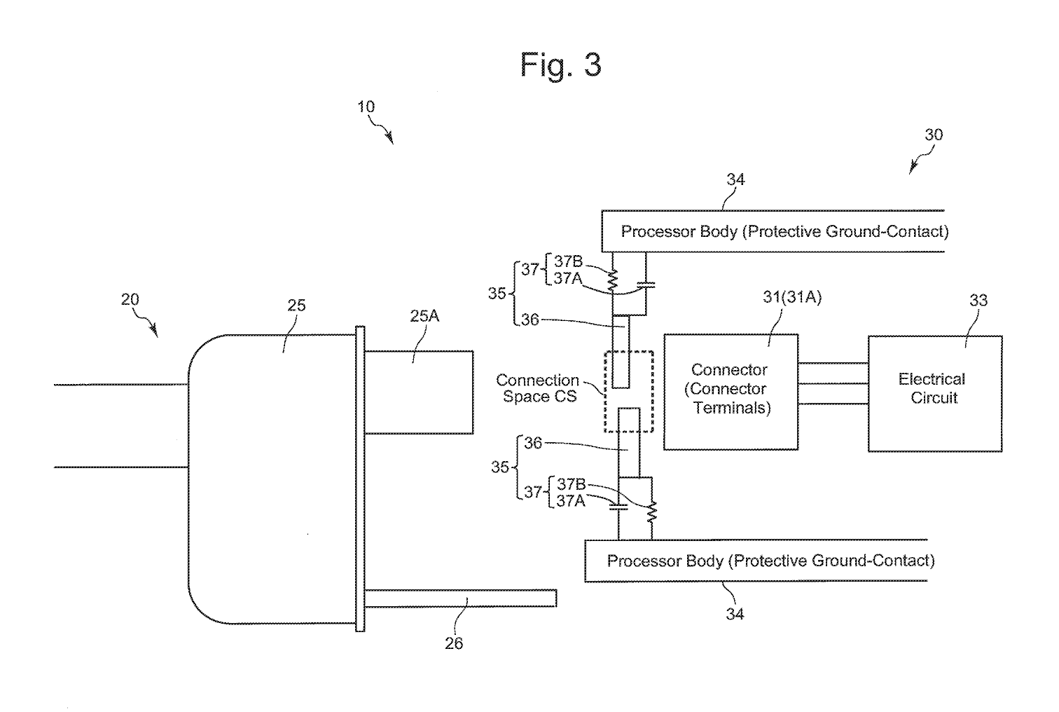

As shown in FIG. 1, the endoscope 20 is provided with a grip control body 21 for the user (operator) to grip onto, and a flexible insertion portion 22 which extends from the grip control body 21. The insertion portion 22 is provided with a distal-end rigid section 22A, a bendable section 22B and a flexible section 22C, in that order from the distal end. The bendable section 22B is bendable in accordance with a rotational operation of a bending-control lever 23, which is provided on the grip control body 21. A universal tube 24 is provided on a side of the grip control body 21 to extend therefrom. A connector (second connector) 25 is provided on the distal end of the universal tube 24, and connector terminals 25A are provided on the end of the connector 25 and project therefrom. Although not shown in the drawings, the endoscope 20 is provided with a built-in light-guide fiber; this light-guide fiber extends from the insertion portion 22 (the distal-end rigid section 22A, the bendable section 22B and the flexible section 22C), the grip control body 21, and the universal tube 24 until extending into a light-guide sleeve 26 which projects from the connector 25. Upon the connector terminals 25A of the connector 25 being connected to connector terminals 31A of a connector 31 provided on the endoscope processor 30 (see FIGS. 2 and 3), the light-guide fiber becomes optically connected to a built-in light-source lamp (not shown in the drawings) provided in the endoscope processor 30.

Furthermore, illumination light that is emitted from the above-mentioned light-source lamp is guided into the light-guide fiber and is externally emitted by a predetermined light distribution via an illumination lens (not shown in the drawings), which is provided at the distal-end face of the distal-end rigid section 22A of the insertion portion 22.

Although not shown in the drawings, the endoscope 20 is provided with an image sensor, positioned within the distal-end rigid section 22A of the insertion portion 22, which obtains observation-image signals of an observation (examined) target site (e.g., a lesion within a patient's body). The observation-image signals obtained by this image sensor is transmitted via a signal-transmission cable (not shown in the drawings) and is output to the endoscope processor 30 via a connection between the connector 25 (connector terminals 25A) and the connector 31 (connector terminals 31A). The endoscope processor 30 is provided with a patient circuit (relay processing circuit) 32 (only shown in FIG. 2; not shown in FIG. 3) which carries out relay processing of the observation signal, and an electrical circuit 33 (only shown in FIG. 3; not shown in FIG. 2) which is configured to carry out a predetermined image processing of the observation signal that was relay-processed by the patient circuit 32. The patient circuit 32 is provided with an electronic component(s) (IC) 32A as a functional component (function block).

Hence, endoscope system 10 can connect the endoscope 20 with the endoscope processor 30 via their respective connectors (the connector terminals 25A of the connector 25 and the connector terminals 31A of the connector 31). In FIG. 2, the connector 31 and the connector terminals 31A of the endoscope processor 30 are depicted as separate blocks that are connected to each other via an electrical connector such as a cable harness, etc.; whereas, in FIG. 3, the connector 31 and the connector terminals 31A of the endoscope processor 30 are depicted as the same block. However, both depictions in FIGS. 2 and 3 signify the connector 31 and the connector terminals 31A being electrically connected to each other.

As shown in FIG. 3, when the connector 25 (connector terminals 25A) of the endoscope 20 is inserted into the endoscope processor 30 to a position immediately in front of the connector 31 (connector terminals 31A) of the endoscope processor 30, there is always a space through which the connector 25 (connector terminals 25A) must pass in order to connect to the connector 31 (connector terminals 31A); this space is herein defined as a “connection space CS” for convenience.

As shown in FIGS. 2 and 3, the endoscope processor 30 is provided with a body 34, into which various components of the endoscope processor 30 are accommodated. This body 34 also functions as a protective ground-contact which is connected to ground.

The endoscope processor 30 is provided with an electrostatic inductor 35, which provided on the body (protective ground-contact) 34 at a positioned immediately in front of the connector 31 (connector terminals 31A). The electrostatic inductor 35 is electrically conductive with the body (protective ground-contact) 34 and is positioned within the connection space CS when the endoscope 20 is not connected to the endoscope processor 30.

More specifically, the electrostatic inductor 35 is provided with a conductive elastic member 36 and a parallel circuit 37.

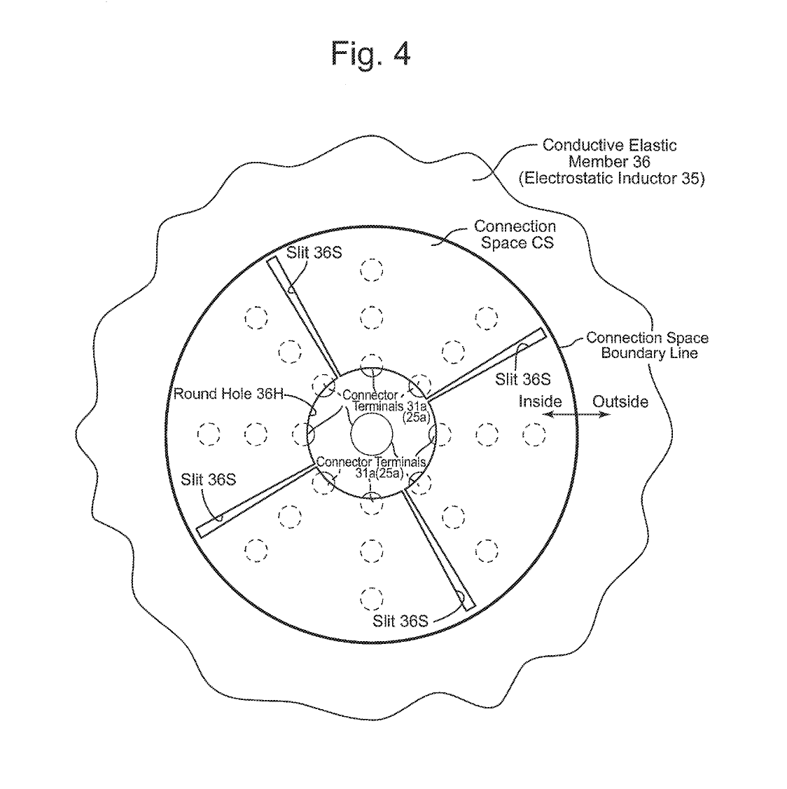

As shown in FIG. 4, the conductive elastic member 36 has a split-curtain shape in a front elevational view (when viewed in a direction facing the endoscope processor 30, opposite to the endoscope 20), in which slits 36S are formed in a central portion and extend from a peripheral edge portion of the round connector 31 (connector terminals 31A) of the endoscope processor 30 toward the central portion (the center of the connector 31). In the example shown in FIG. 4, four slits 36S are formed in the conductive elastic member 36 at 90° intervals in a circumferential direction of an annular member, in which a round hole 36H is formed at the center. The conductive elastic member 36 can be formed from a material through which an instantaneous charge can be discharged (impulse) and through which a DC current can be passed; however, there is a certain amount of freedom in the material that can be used for the conductive elastic member 36, and various design changes are possible.

As shown in FIG. 4, the conductive elastic member 36 is in a free state when the endoscope 20 and the endoscope processor 30 are not connected to each other, and the conductive elastic member 36 enters inside the boundary of the connection space CS to almost cover the connector 31 (connector terminals 31A) of the endoscope processor 30. Whereas, when the connector portion 25 (connector terminals 25A) is brought close to the connector 31 (connector terminals 31A) in order to connect the endoscope 20 to the endoscope processor 30, due to the connector 25 (connector terminals 25A) of the endoscope 20 coming in contact with the conductive elastic member 36, the conductive elastic member 36 deforms (elastically deforms), starting from the slits 36S, toward the outside the boundary of the connection space CS and toward the endoscope processor 30.

The parallel circuit 37 is configured of a capacitor 37A and a resistor 37B, and by connecting the conductive elastic member 36 with the body (protective ground-contact) 34 via the parallel circuit 37, an impedance can be obtained between the conductive elastic member 36 and the body (protective ground-contact) 34. The constant of the capacitor 37A and the resistor 37B of the parallel circuit 37 is chosen so that a certain amount of impedance is obtained with respect to the body (protective ground-contact) 34. For example, if the capacitor 37A was set at 100 pF and the resistor 37B was set at 10 MQ, a time constant TC can be calculated as tr=0.02 sec. Accordingly, the parallel circuit 37 can be practically used to sufficiently withstand a discharge time period. Although not shown in FIG. 4, the parallel circuit 37 is provided on the outer side of the boundary line of the connection space CS and along the peripheral direction of the conductive elastic member 36.

As shown in FIG. 2, the endoscope processor 30 is provided with an impedance component (capacitor) 38 connecting the patient circuit 32 with the body (protective ground-contact) 34. Hence, accumulated charge from the patient circuit 32 can be discharged to the body (protective ground-contact) 34 via the impedance component 38. The amount of discharge (electrostatic protective capacity) via the impedance component 38 is set much smaller than the amount of discharge (electrostatic protective capacity) of the electrostatic inductor 35.

The connecting operation of endoscope system (endoscope connection system) 10 having the above configuration will be described hereinbelow.

As shown in FIGS. 3 and 4, in a state where the endoscope 20 is not connected to the endoscope processor 30, the conductive elastic member 36 of the electrostatic inductor 35 is in a free state and positioned inside the connection space CS, so that the connector 31 (the connector terminals 31A) of the endoscope processor 30 is almost completely covered.

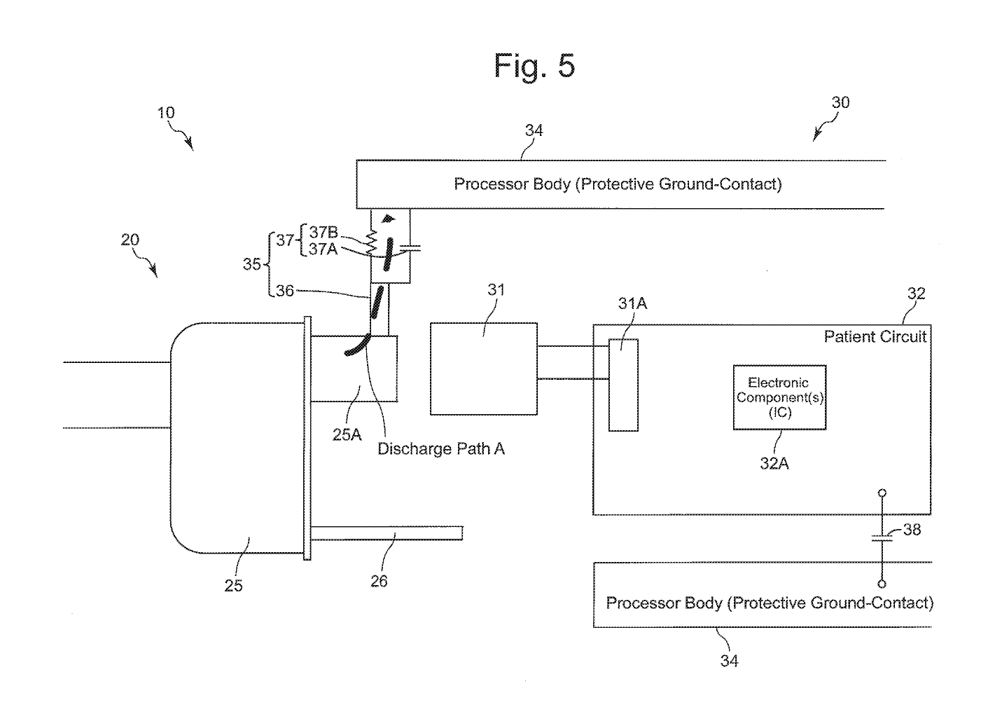

As shown in FIG. 5, when the connector 25 (connector terminals 25A) is brought close to the connector 31 (connector terminals 31A) in order to connect the endoscope 20 to the endoscope processor 30, the connector 25 (connector terminals 25A) of the endoscope 20 first contacts the conductive elastic member 36. At this stage, static electricity (electrostatic charge) that is accumulated on the connector 25 (connector terminals 25A) of the endoscope 20 is discharged to the body (protective ground-contact) 34 (discharge path A) via the electrostatic inductor 35 (the conductive elastic member 36 and the parallel circuit 37).

Furthermore, upon the connector 25 (connector terminals 25A) being brought close to the connector 31 (connector terminals 31A), the conductive elastic member 36 moves outside the connection space CS due to the conductive elastic member 36 deforming (elastically deforming) while maintaining a contacted state with the connector 25 (connector terminals 25A) of the endoscope 20. In other words, the conductive elastic member 36 is configured so as not to hinder the connection between the connector 25 (connector terminals 25A) and the connector 31 (connector terminals 31A).

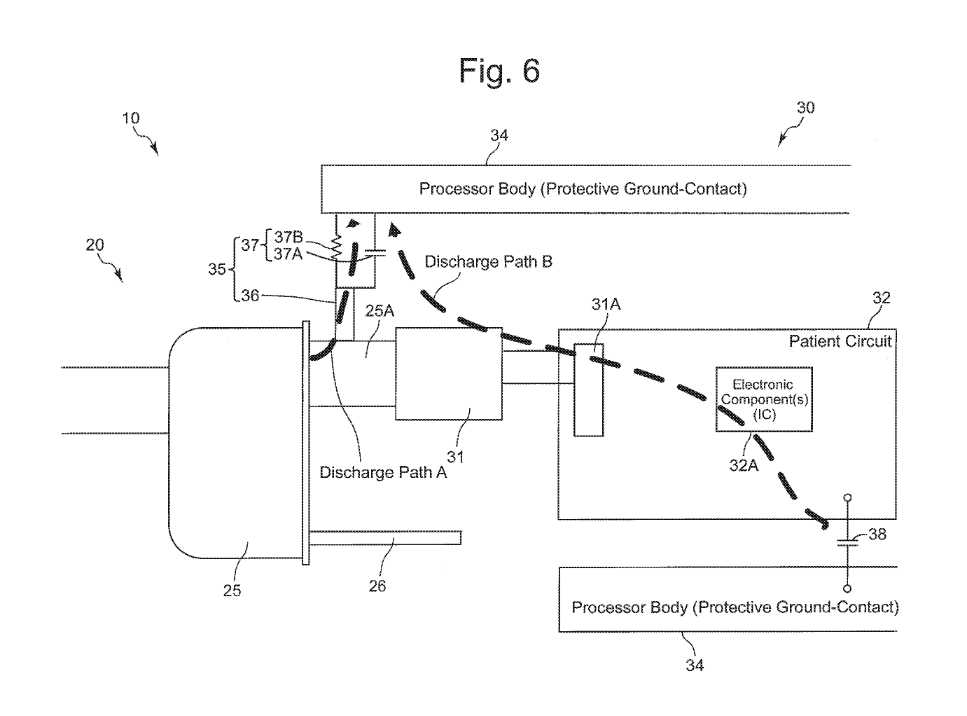

FIG. 6 shows a state where the connector 25 (connector terminals 25A) of the endoscope 20 is connected to the connector 31 (connector terminals 31A) of the endoscope processor 30. As shown in FIG. 6, since the conductive elastic member 36 is always in contact with the connector 25 (connector terminals 25A) of the endoscope 20, a large electrostatic charge does not accumulate in the endoscope processor 30, and even if certain amount of charge were to accumulate, such a charge would quickly discharge via a discharge path B. Accordingly, it is possible to protect the electronic component(s) (IC) 32A of the patient circuit 32 by preventing an excessively large amount of discharge current from discharging therethrough.

Hence, when the connector 25 (connector terminals 25A) of the endoscope 20 is brought close to be connected to the connector 31 (connector terminals 31A) of the endoscope processor 30, the electrostatic inductor 35 (conductive elastic member 36) contacts the connector 25 (connector terminals 25A) of the endoscope 20 in the connection space CS before the connectors 25 and 31 are connected to each other, and also continues to remain in contact with the connector 25 (connector terminals 25A) of the endoscope 20 after the connectors 25 and 31 have connected with each other. Accordingly, an electrostatic charge can escape (discharge) via the discharge path A before the connectors 25 and 31 are connected to each other, and an electrostatic charge can escape (discharge) via discharge path A and discharge path B after the connectors 25 and 31 are connected to each other.

Whereas, in an endoscope connection system of the related art, in a state where an electrostatic charge has accumulated in the patient circuit of an endoscope processor due to continuous electrostatic charging, if insulation breakdown occurs in an electrostatic protection mechanism, during the instant the insulation breakdown occurs, the electrostatic charge accumulated in the patient circuit flows to the protective ground contact of the endoscope all at once. Accordingly, there is a high risk of electrostatic breakdown occurring in the electronic component (IC) in the patient circuit.

However, in a state where the endoscope 20 is not connected with the endoscope processor 30, it is possible (conceivable) for a user's hand, etc., that is electrostatically charged to closely approach the connector 31 (connector terminals 31A) of the endoscope processor 30. In such a case, in an endoscope connection system of the related art, there is a risk of an electrostatic charge discharging to the connector (connector terminals) of the endoscope processor, thereby applying an unintentional load on an electronic component(s) in the endoscope processor.

Whereas, in the endoscope system (endoscope connection system) 10 of the illustrated embodiment, even if a user's hand, etc., that is electrostatically charged closely approaches the connector 31 (connector terminals 31A) of the endoscope processor 30 when the endoscope 20 and the endoscope processor 30 are not connected to each other, the user's hand, etc., would first contact the conductive elastic member 36 of the electrostatic inductor 35. Therefore, since the electrostatic charge discharges to the body (protective ground-contact) 34 via the electrostatic inductor 35 (the conductive elastic member 36 and the parallel circuit 37), the above-described technical problems of the endoscope connection system of the related art can be solved. Namely, an electrostatic load on the electronic component(s) (IC) 32A of the patient circuit 32 of the endoscope processor 30 can be effectively prevented.

It is important to note that, due to the standard stipulated for medical devices which states that the patient circuit 32 of an endoscope processor 30 must not be directly connected to ground, there is a tendency for electrostatic protection to be insufficient (vulnerable); hence, an effective countermeasure is required. The endoscope system (endoscope connection system) 10 of the illustrated embodiment is useful for effective electrostatic protection of the patient circuit 32 while strictly adhering to the medical device standard which states that the patient circuit 32 must not be directly connected to ground.

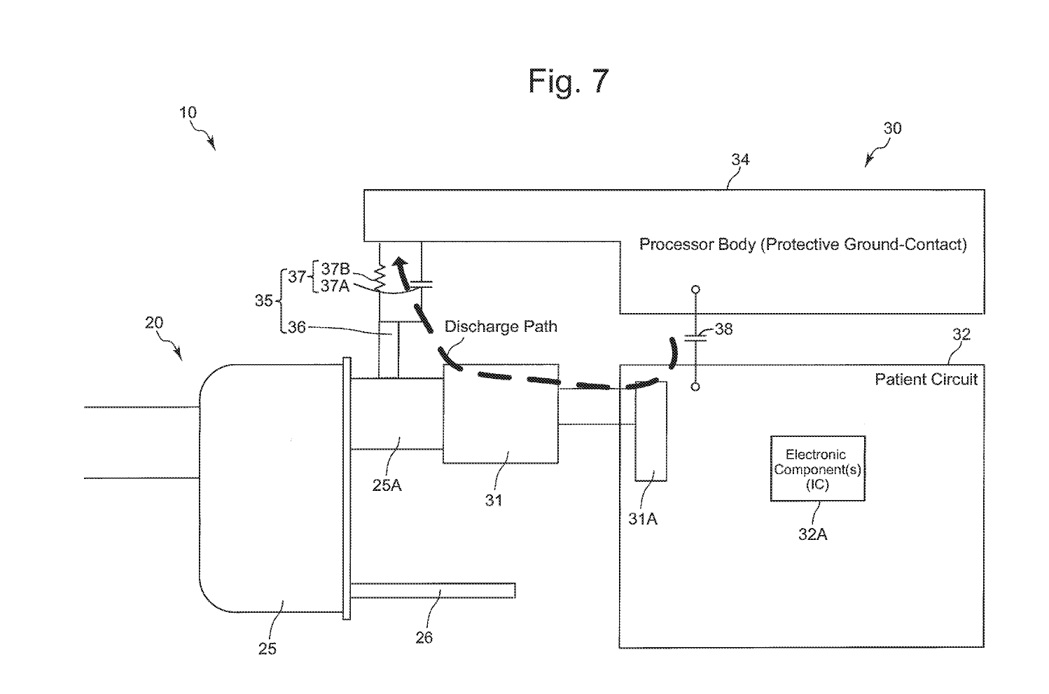

FIG. 7 shows a second embodiment of the endoscope system (endoscope connection system) 10. In a state where a large amount of charge has accumulated in the patient circuit 32 of the endoscope processor 30 due to some reason, if the endoscope 20 is connected to the endoscope processor 30, a certain amount of electrostatic discharge current flows to the patient circuit 32. If the electronic component(s) (IC) 32A is located in the discharge path of this flow of electrostatic discharge current, there is a risk of the electronic component(s) (IC) 32A being damaged (in the embodiment shown in FIG. 6, the electronic component(s) (IC) 32A is located in the discharge path B).

In the second embodiment, the impedance component (capacitor) 38 is provided at a location that allows the electronic component(s) (IC) 32A of the patient circuit 32 to avoid the discharge path that flows from the patient circuit 32 to the body (protective ground-contact) 34. Out of the patient circuit 32 of the endoscope processor 30, the impedance component (capacitor) 38, which is a secondary circuit, holds most of the electrostatic charge. Hence, electrostatic breakage of the electronic component(s) (IC) 32A can be prevented by appropriately determining an optimum position of the impedance component 38 so that the electronic component(s) (IC) 32A of the patient circuit 32 does not coincide with the electrostatic discharge path.

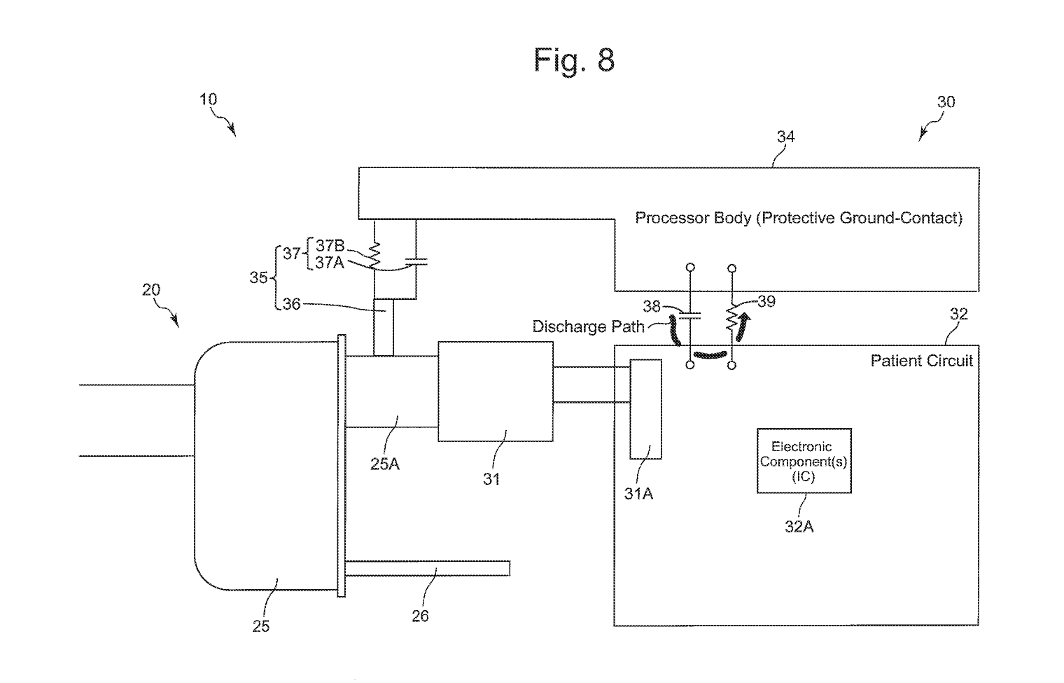

FIG. 8 shows a third embodiment of the endoscope system (endoscope connection system) 10. In the third embodiment, in addition to the configuration of FIG. 7, the endoscope processor 30 is provided with a discharge resistor 39 arranged in parallel with the impedance component (capacitor) 38 and connecting between the patient circuit 32 and the body (protective ground-contact) 34. According to this configuration, it is possible for electrostatic charge to escape to the body (protective ground-contact) 34 via the discharge resistor 39 without accumulating at the impedance component 38. The resistance value of the discharge resistor 39 is set to a constant that is determined in accordance with electrical safety.

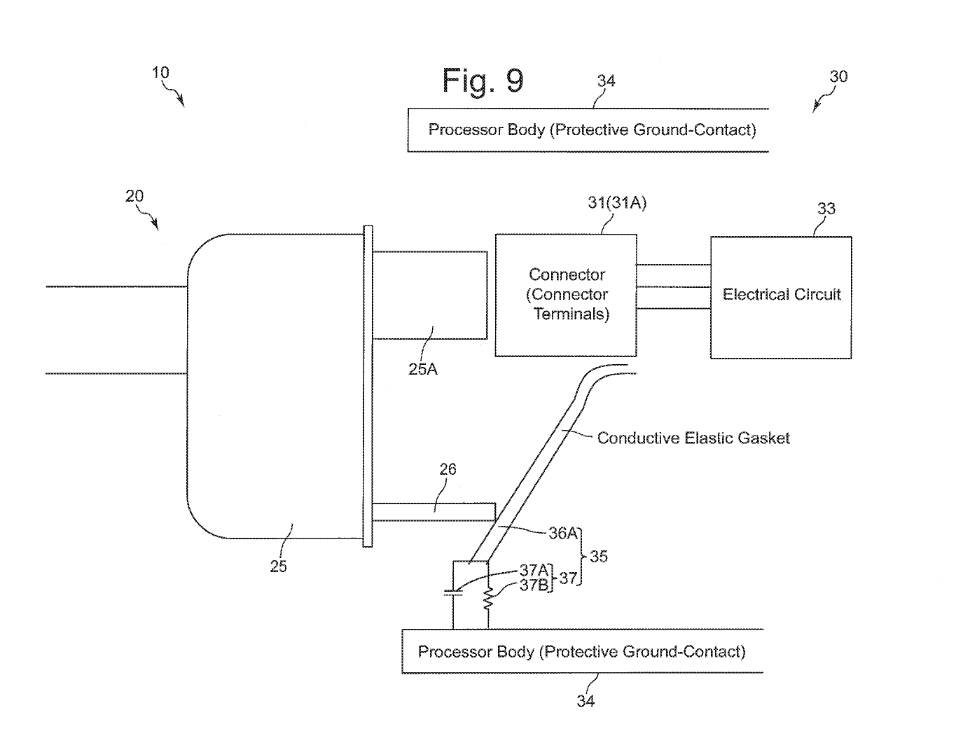

FIG. 9 shows a fourth embodiment of the endoscope system (endoscope connection system) 10. In the fourth embodiment, a conductive elastic gasket 36A is provided as (instead of) the conductive elastic member 36 of the arrangements shown in FIGS. 1 through 8 (first through third embodiments). The conductive elastic gasket 36A can elastically deform in both a connecting direction between the connector 25 (connector terminals 25A) of the endoscope 20 and the connector 31 (connector terminals 31A) of the endoscope processor 30, and in a direction orthogonal to the connecting direction.

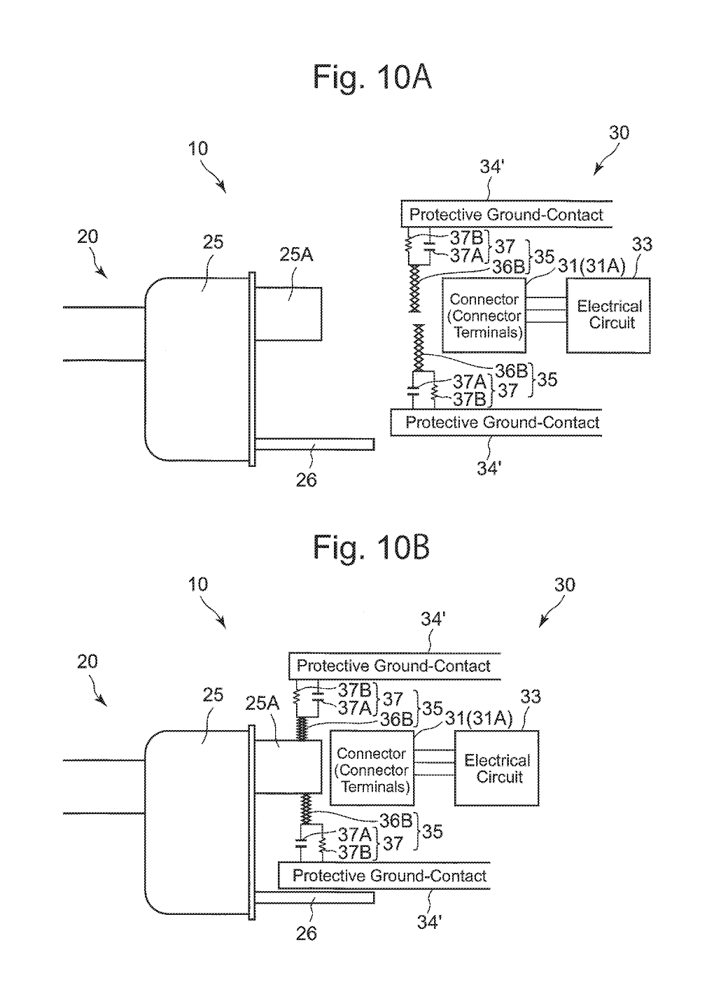

FIGS. 10A and 10B show a fifth embodiment of the endoscope system (endoscope connection system) 10. FIG. 10A shows a state where the endoscope 20 is not connected (separated state) to the endoscope processor 30, and FIG. 10B shows a state immediately before the endoscope 20 is connected to the endoscope processor 30. In the fifth embodiment, conductive elastic compression springs 36B are provided as (instead of) the conductive elastic member 36 of the arrangements shown in FIGS. 1 through 8 (first through third embodiments), and instead of the conductive elastic gasket 36A of the fourth embodiment. The conductive elastic compression springs 36B are elastically deformable (compressible) in only a direction that is orthogonal to the connecting direction between the connector 25 (connector terminals 25A) of the endoscope 20 and the connector 31 (connector terminals 31A) of the endoscope processor 30. Furthermore, in the fifth embodiment, a protective ground-contact 34′ is provided on an inner side of the body 34 as a separate component from that of the body 34.

In the above-described first through fifth embodiments, an example has been described in which the electrostatic inductor 35 (conductive elastic member 36, conductive elastic gasket 36A or conductive elastic compression springs 36B) contacts the connector 25 (connector terminals 25A) of the endoscope 20 when the connector 25 (connector terminals 25A) of the endoscope 20 is brought close to the connector 31 (connector terminals 31A) of the 30 before being connected, and the electrostatic inductor 35 (conductive elastic member 36, conductive elastic gasket 36A or conductive elastic compression springs 36B) also continues to contact the connector 25 (connector terminals 25A) of the endoscope 20 after the connectors 25 and 31 are connected to each other. However, a configuration is possible in which the electrostatic inductor 35 (conductive elastic member 36, conductive elastic gasket 36A or conductive elastic compression springs 36B) only contacts the connector 25 (connector terminals 25A) of the endoscope 20 before the connectors 25 are 31 connect with each other, and the electrostatic inductor 35 (conductive elastic member 36, conductive elastic gasket 36A or conductive elastic compression springs 36B) retracts (moves away) from the connector 25 (connector terminals 25A) of the endoscope 20 after the connectors 25 and 31 connect with each other to release the contacting state between the electrostatic inductor 35 (conductive elastic member 36, conductive elastic gasket 36A or conductive elastic compression springs 36B) and the connector 25 (connector terminals 25A).

In the above-described embodiments, an example is given (in FIG. 4) in which the conductive elastic member 36 (conductive elastic gasket 36A or conductive elastic compression springs 36B) is provided with four slits arranged at four 90° intervals on the annular member, the center at which a circular hole is formed. However, there is a certain amount of freedom in regard to the shape of the conductive elastic member 36; various design changes are possible. In other words, the conductive elastic member 36 is only required to be positioned in the connection space CS when the endoscope 20 and the endoscope processor 30 are not connected to each other (when the conductive elastic member 36 is in a free state), and the conductive elastic member 36 is only required to first contact the connector 25 (connector terminals 25A) of the endoscope 20 when the connector 25 (connector terminals 25A) is brought close to the connector 31 (connector terminals 31A) when the endoscope 20 is being connected to the endoscope processor 30.

In the above-described embodiments, the connector 25 (connector terminals 25A) of the endoscope 20 and the connector 31 (connector terminals 31A) of the endoscope processor 30 are round in shape, however, the present invention is not limited thereto; various other shaped connectors (connector terminals) are possible, such as a polygonal shape.

### REFERENCE SIGNS LIST

10 Endoscope system
20 Endoscope
21 Grip control body
22 Insertion portion
22A Distal-end rigid section
22B Bendable section
22C Flexible section
23 Bending-control lever
24 Universal tube
25 Connector
25A Connector terminals
26 Light-guide sleeve
30 Endoscope processor
31 Connector
31A Connector terminals (connector)
32 Patient circuit
32A Electronic component(s) (IC)
33 Electrical circuit
34 Body (protective ground-contact)
34′ Protective ground-contact
35 Electrostatic inductor
36 Conductive elastic member
36A Conductive elastic gasket
36B Conductive elastic compression springs
36H Round hole
36S Slits
37 Parallel circuit
37A Capacitor
37B Resistor
38 Impedance component (capacitor)
39 Discharge resistor
CS Connection space

1. An endoscope processor comprising:
a connector configured to connect with a second connector, provided on an endoscope;
a protective ground-contact connected to ground; and
an electrostatic inductor connected to, and electrically conductive with, said protective ground-contact, said electrostatic inductor positioned in a connection space between said connector and said second connector when said endoscope and said endoscope processor are not connected to each other.

2. The endoscope processor according to claim 1, wherein, when said connector of said endoscope processor and said second connector of said endoscope and are brought close to each other to be connected to each other, said electrostatic inductor is configured to contact said second connector of said endoscope in said connection space before said connector and said second connector are connected to each other, and said electrostatic inductor is configured to continue to be in contact with said second connector of said endoscope upon said connector and said second connector being connected to each other.

3. The endoscope processor according to claim 2, wherein said electrostatic inductor is configured to continue to be in contact with said second connector of said endoscope by deforming upon said electrostatic inductor contacting with said second connector.

4. The endoscope processor according to claim 1, wherein said electrostatic inductor comprises:
a conductive elastic member extending from an outer periphery toward a center of said connector of said endoscope processor; and
a parallel circuit including a capacitor and a resistor which are each connected between said conductive elastic member and said protective ground-contact.

5. The endoscope processor according to claim 1, further comprising:
a patient circuit provided with at least one electronic component; and
an impedance component connected between said patient circuit and said protective ground-contact,
wherein said endoscope processor is configured so that an accumulated electrostatic charge in said patient circuit discharges to said protective ground-contact via said impedance component, and
wherein said impedance component is arranged so that said electronic component of said patient circuit avoids a discharge path that flows from said patient circuit to said protective ground-contact.

6. The endoscope processor according to claim 5, further comprising a discharge resistor connected between said patient circuit and said protective ground-contact, said discharge resistor arranged in parallel with said impedance component.

7. The endoscope processor according to claim 1, wherein said protective ground-contact is provided on a body of said endoscope processor, and
wherein said electrostatic inductor is provided on said protective ground-contact at a position immediately in front of said connector of said endoscope processor.

8. An endoscope connection system configured to connect an endoscope to an endoscope processor via a connector and a second connector, wherein said endoscope processor comprises:
a protective ground-contact connected to ground; and
an electrostatic inductor connected to, and electrically conductive with, said protective ground-contact, said electrostatic inductor positioned in a connection space between said connector and said second connector when said endoscope and said endoscope processor are not connected to each other.
